(19) United States
(12) Patent Application Publication
(10) Pub. No.: US 2025/0354969 A1
(43) Pub. Date: Nov. 20, 2025

(54) DEVICES AND METHODS FOR AIR QUALITY MONITORING

(71) Applicant: Synthetik Applied Technologies, LLC, Austin, TX (US)

(72) Inventors: Damon Cardenas, Austin, TX (US); David Memke, Austin, TX (US); Edward Acosta, Austin, TX (US); Karyn Vo, Austin, TX (US); Michael Pyle, Austin, TX (US); Hannah Stevens, Austin, TX (US)

(21) Appl. No.: 18/667,333

(22) Filed: May 17, 2024

(51) Int. Cl. G01N 33/00; G01V 11/00

(52) U.S. Cl. CPC G01N 33/0075; G01N 33/0062; G01V 11/002

(57) ABSTRACT

The present disclosure relates generally to air quality and occupancy monitoring (AQOM) system that include at least one AQOM monitoring device and a portal configured to communicate with the monitoring devices. The monitoring devices have sensors that collect and send environmental data to the portal. The portal, in turn, allows a user to access, review, analyze, and/or download the environmental data collected. In some embodiments of the AQOM monitoring device, the sensors are configured on an interchangeable component allowing for modularity of the device.

### RELATED APPLICATION

The present application claims priority to U.S. Provisional Patent Application No. 63/502,878 by Cardenas, et al., entitled DEVICES AND METHODS FOR AIR QUALITY MONITORING filed on May 17, 2023. This provisional application is fully incorporated by reference herein in its entirety.

### BACKGROUND OF THE DISCLOSURE

### Field of the Disclosure

The present disclosure relates generally to systems, devices, and methods for collecting, viewing, and analyzing air quality, occupancy, and other environmental data.

### Description of the Related Art

Air quality, occupancy, and environmental monitoring is key to maintaining human safety and comfort in a variety of settings, such as hospitals, manufacturing plants, power plants, entertainment venues, and so on. It is important to be able to determine when air quality declines so as to require evacuation or ventilation. Monitoring solutions exist for air quality, occupancy, or environmental variables but cost-effective, aesthetic, and easily deployable solutions do not exist for monitoring and data synthesizing for all three categories simultaneously. Moreover, the monitoring devices available must be wired to or plugged into their power source, or, if battery powered, the battery has limited working life and is not rechargeable or replaceable.

### SUMMARY OF THE DISCLOSURE

One embodiment of an air quality and occupancy monitoring (AQOM) system according to the present disclosure includes (1) at least one monitoring device and (2) a portal configured to communicate with the monitoring device or devices. The monitoring device has one or more sensors that collect environmental data and the sensors in turn send that data to the portal. The portal then allows a user to access, review, analyze, and/or download the data collected. In a preferred embodiment, the devices and portal wirelessly communicate with one another. However, wired communication is also possible. In some embodiments, the portal also communicates with the monitoring device, and can send information, instructions, or updates to the monitoring device.

One embodiment of an AQOM device according to the present disclosure includes (1) a body, (2) a printed circuit board configured to be housed in the body, (3) at least one sensor also configured to be housed in the body, and (4) a power source connected to the printed circuit board and sensor. The sensors are able to measure and collect environmental information. There are many options for a power source, such as a battery, solar, or electrical.

In some embodiments of an AQOM device according to the present disclosure, the battery is replaceable and/or rechargeable. Also, in some embodiments of an AQOM device according to the present disclosure, the sensor or sensors are located on a sensor suite that is removable from the device and interchangeable with other sensor suites.

The systems and devices according the present disclosure are customizable, lightweight, aesthetically pleasing, mountable, and easily deployable.

This has outlined, rather broadly, the features and technical advantages of the present disclosure so that the detailed description that follows may be better understood. Additional features and advantages of the disclosure will be described below. It should be appreciated by those skilled in the art that this disclosure may be readily utilized as a basis for modifying or designing other structures or systems for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further features and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

FIG. 1A shows a top right perspective view of an embodiment of an AQOM device incorporating features of the present disclosure.

FIG. 1B shows a top view of the embodiment of FIG. 1A.

FIG. 1C shows a bottom view of the embodiment of FIG. 1A.

FIG. 1D shows a right side view of the embodiment of FIG. 1A.

FIG. 1E shows a left side view of the embodiment of FIG. 1A.

FIG. 2 shows an exploded view of the embodiment of FIG. 1A.

FIG. 3A shows a top right perspective view of a second embodiment of an AQOM device incorporating features of the present disclosure.

FIG. 3B shows a front view of the embodiment of FIG. 3A;

FIG. 3C shows a back view of the embodiment of FIG. 3A;

FIG. 3D shows a top view of the embodiment of FIG. 3A.

FIG. 3E shows a bottom view of the embodiment of FIG. 3A.

FIG. 3F shows a right side view of the embodiment of FIG. 3A.

FIG. 3G shows a left side view of the embodiment of FIG. 3A.

FIG. 4 shows an exploded view of the embodiment of FIG. 3A.

FIG. 5A shows a top right perspective view of a third embodiment of an AQOM device incorporating features of the present disclosure.

FIG. 5B shows a front view of the embodiment of FIG. 5A;

FIG. 5C shows a back view of the embodiment of FIG. 5A;

FIG. 5D shows a right side view of the embodiment of FIG. 5A;

FIG. 5E shows a left side view of the embodiment of FIG. 5A;

FIG. 6 shows a top right perspective view of the embodiment of FIG. 5A connected to a junction box;

FIGS. 7A-7K are examples of user portal configurations according to an embodiment of the present disclosure.

FIGS. 8A-8B are examples of user portal configurations according to a second embodiment of the present disclosure.

### DETAILED DESCRIPTION OF THE DISCLOSURE

This disclosure relates generally to air quality, environment, and occupancy safety and monitoring. More particularly, this disclosure relates to devices and systems for analyzing and monitoring air quality, environmental conditions, and occupancy.

In the following detailed description, numerous specific details are set forth in order to provide a more thorough understanding of embodiments incorporating features of the present disclosure. However, it will be apparent to one skilled in the art that systems and devices according to the present disclosure can be practiced without necessarily being limited to these specifically recited details.

Throughout this disclosure, the embodiments illustrated and the components therein, including, but not limited to, specific microcontrollers, power sources, and sensors, should be considered as exemplars, rather than as limitations on the present disclosure. As used herein, the term “composition,” “device,” “structure,” “method,” “system,” “disclosure,” “present composition,” “present device,” “present structure,” “present method,” “present system,” or “present disclosure” refers to any one of the embodiments of the disclosure described herein, and any equivalents. Furthermore, reference to various feature(s) of the “composition,” “device,” “structure,” “method,” “system,” “disclosure,” “present composition,” “present device,” “present structure,” “present method, “present system,” “present apparatus,” or “present disclosure” throughout this document does not mean that all claimed embodiments or methods must include the reference feature(s).

Furthermore, any element in a claim that does not explicitly state “means for” performing a specified function, or “step for” performing a specific function, is not to be interpreted as a “means” or “step” clause as specified in 35 U.S.C. § 112, for example, in 35 U.S.C. § 112(f) or pre-AIA 35 U.S.C. § 112, sixth paragraph. In particular, the use of “step of” or “act of” in the claims herein is not intended to invoke the provisions of 35 U.S.C. § 112.

It is also understood that when an element or feature is referred to as being “on” or “adjacent” to another element or feature, it can be directly on or adjacent the other element or feature or intervening elements or features may also be present. It is also understood that when an element is referred to as being “attached,” “connected” or “coupled” to another element, it can be directly attached, connected or coupled to the other element or intervening elements may be present. In contrast, when an element is referred to as being “directly attached,” “directly connected” or “directly coupled” to another element, there are no intervening elements present.

Furthermore, relative terms such as “left,” “right,” “front,” “back,” “top,” “bottom’” “forward,” “reverse,” “clockwise,” “counter-clockwise,” “outer,” “inner,” “above,” “upper,” “lower,” “below,” “horizontal,” “vertical,” and similar terms, have been used for convenience purposes only and are not intended to imply any particular fixed direction. Instead, they are used to describe a relationship of one element to another. Terms such as “higher,” “lower,” “wider,” “narrower,” and similar terms, may be used herein to describe angular relationships. It is understood that these terms are intended to encompass different orientations of the elements or system in addition to the orientation depicted in the figures.

Although ordinal terms, e.g., first, second, third, etc., may be used herein to describe various elements or components, these elements or components should not be limited by these terms. These terms are only used to distinguish one element or component from another element or component. Thus, a first element or component discussed below could be termed a second element or component without departing from the teachings of the present disclosure.

The terminology used herein is for describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” when used herein, specify the presence of stated features, integers, steps, operations, elements, and/or components, but do not preclude the presence or addition of one or more other features, integers, steps, operations, elements, components, and/or groups thereof.

Embodiments as described in the present disclosure can be described herein with reference to view illustrations that are schematic in nature. As such, the actual thickness of elements can be different, and variations from the shapes of the illustrations as a result, for example, of manufacturing techniques and/or tolerances are expected. Thus, the elements illustrated in the figures are schematic in nature and their shapes are not intended to illustrate the precise shape of a region and are not intended to limit the scope of the disclosure. Further, it is understood that, while embodiments of the present disclosure comprise various shapes, these shapes are not exhaustive, and other shapes are possible.

It is understood that when a first element is referred to as being “between” or “interposed between” two or more other elements, the first element can be directly between the two or more other elements or intervening elements may also be present between the two or more other elements. For example, if a first element is “between” or “interposed between” a second and third element, the first element can be directly between the second and third elements with no intervening elements, or the first element can be adjacent to one or more additional elements with the first element and these additional elements all between the second and third elements.

### The AQOM Device

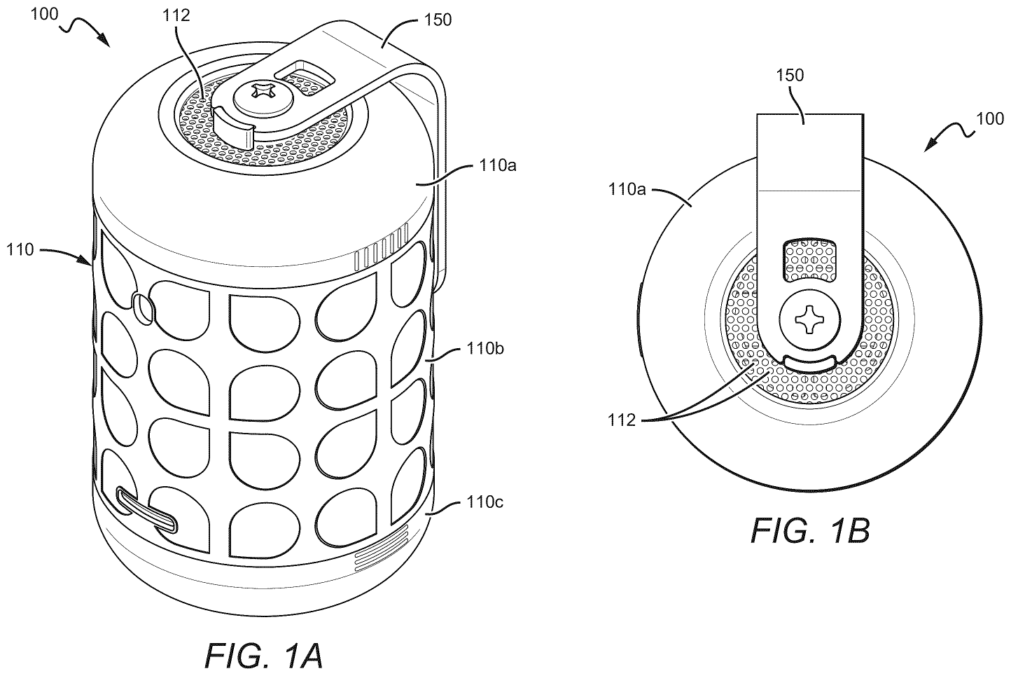

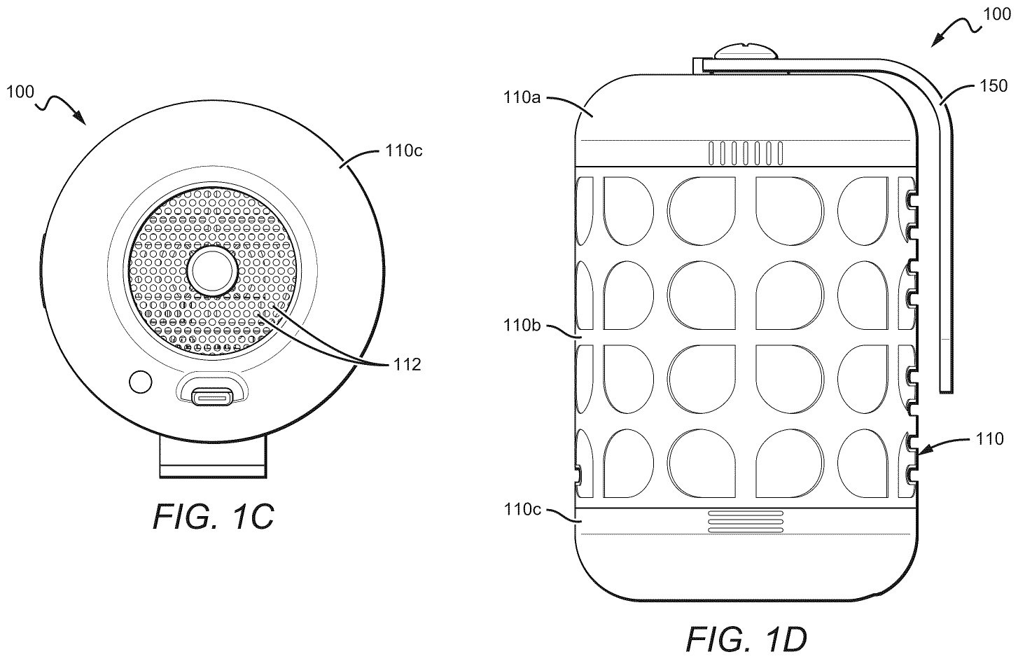

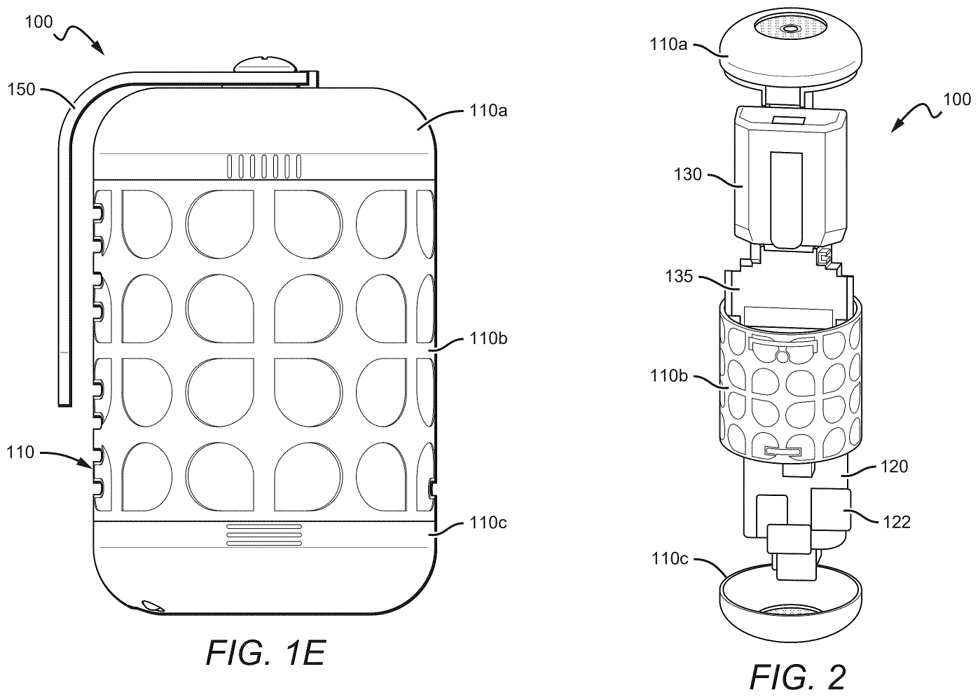

FIGS. 1A-1E and 2 show one embodiment of an AQOM device 100 according to the present disclosure. The device 100 shown in FIGS. 1A-1E and 2, comprises a body 110, a printed circuit board 120 housed inside the body 110, and a power source 130 that is connected to the body 110 and the printed circuit board 120. As best seen in FIG. 2, sensors 122 are connected or attached to the printed circuit board 120. At least one sensor 122 should be connected or attached to the printed circuit board 120. Also, in FIG. 2, the power source is a battery 130 is connected to and powers the device via a connection mechanism 135.

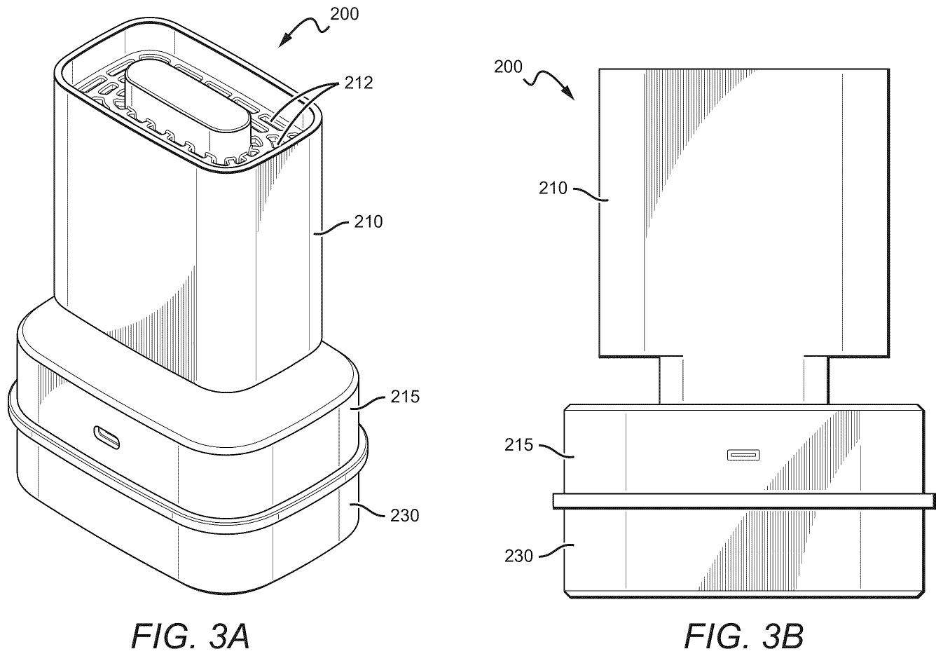

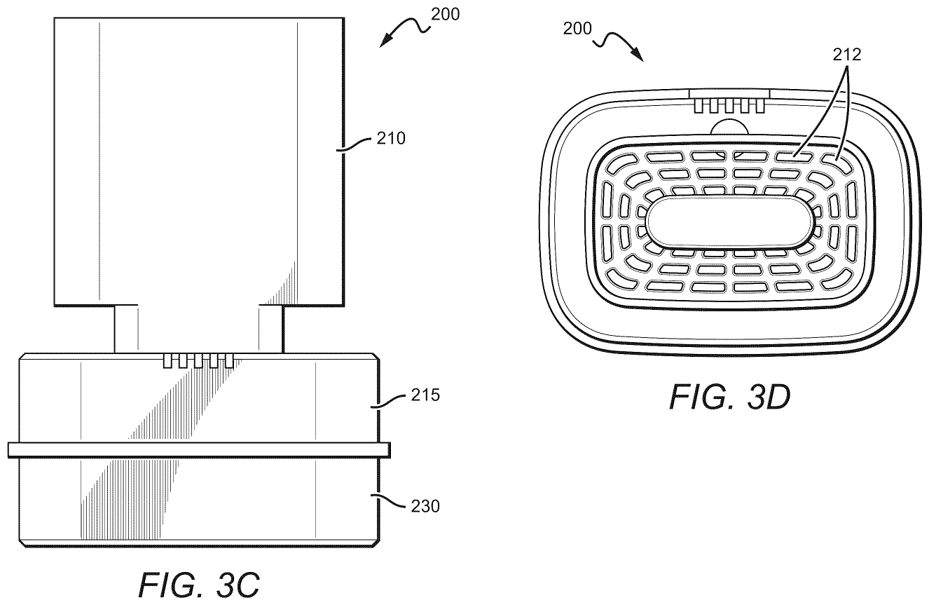

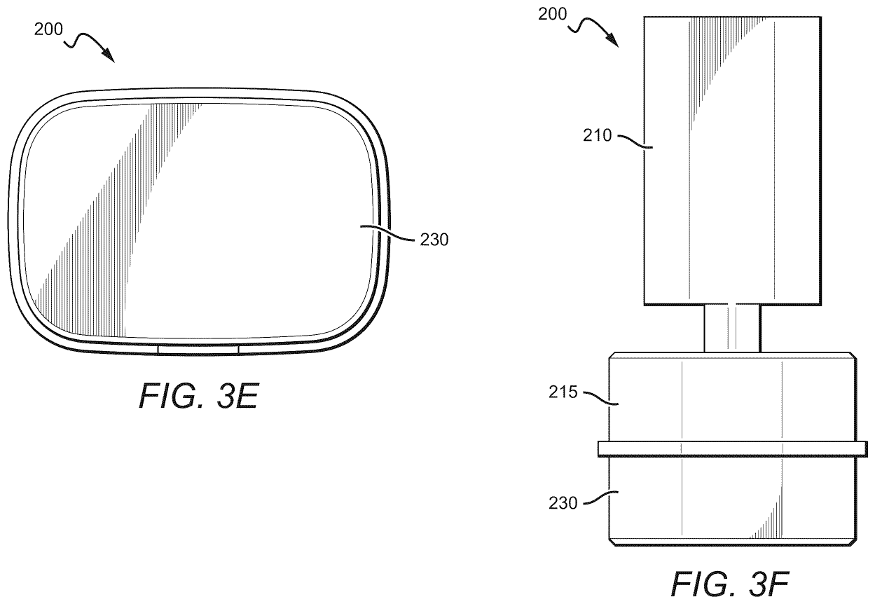

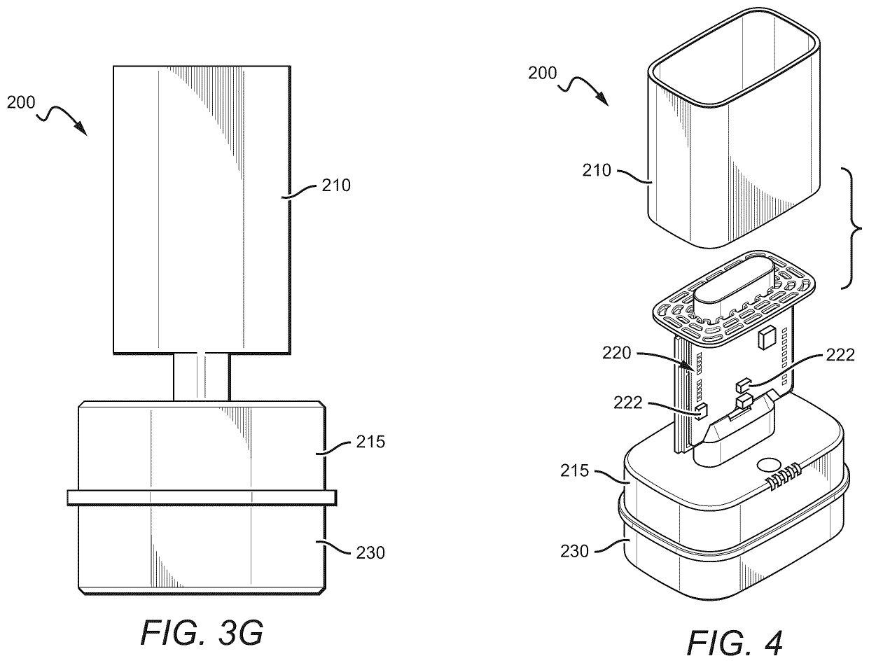

FIGS. 3A-3G and 4 show another embodiment of an AQOM device 200 according to the present disclosure. As best exemplified in FIG. 4, the device 200 shown comprises a body 210, at least one sensor 222 housed inside the body 210, a printed circuit board housed inside a protective container 215, and a power source 230. As shown, the sensors 222 can be configured to be attached to a sensor suite 220. In the embodiment shown, the power source is a battery 230 that may be removably connected to the circuit board 215, the sensor suite, 220, and the body 210.

The sensor suite 220 comprises at least one sensor 222. The sensor suite 220 may be easily interchangeable with different sensor suites comprised of different sensors, making the device 200 modular and customizable. The ability to remove the sensor suite 220 also allows for streamlined repair.

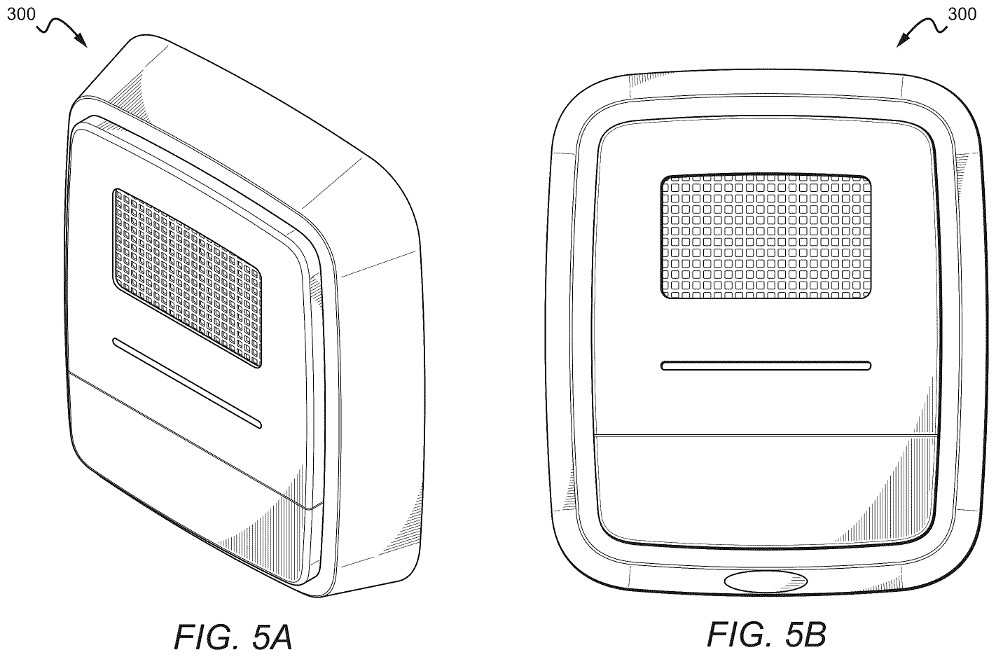

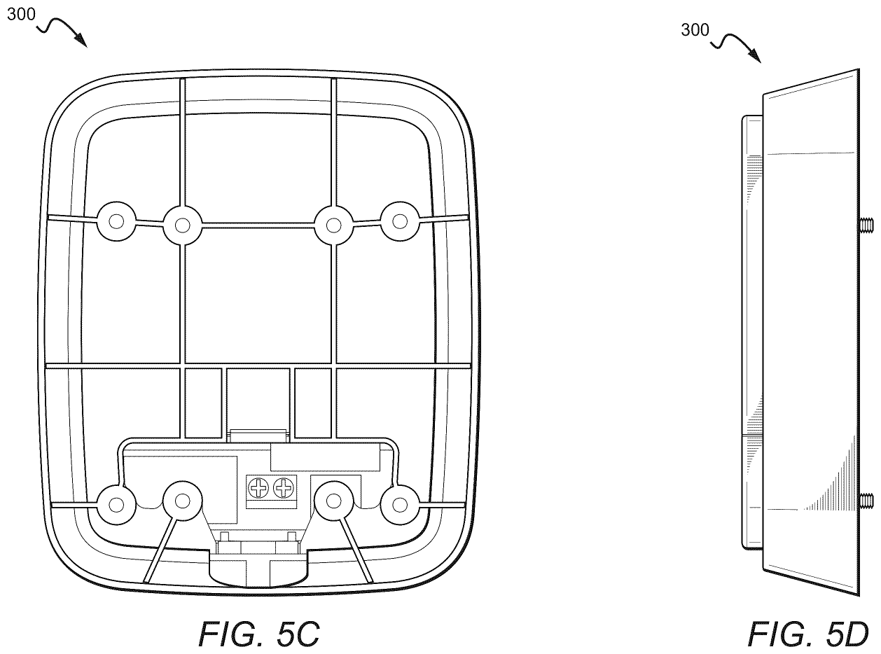

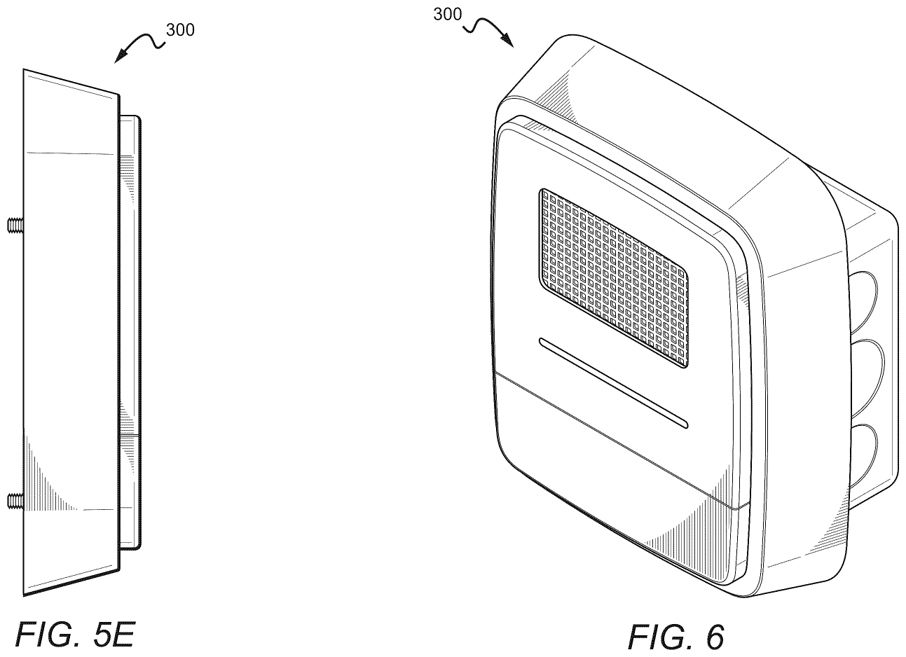

FIGS. 5A-5E and 6 show a third embodiment of an AQOM device 300 according to the present disclosure. Like the embodiments shown in FIGS. 1A-1E, 2, 3A-3G, and 4, the device 300 shown comprises a body, at least one sensor, a printed circuit board, and a power source connected to the body, the sensor(s), and the printed circuit board. The sensors in this embodiment may also be configured on a sensor suite that is interchangeable. The sensor suite in an AQOM device 300 may be attached to a removable front panel of the device promoting easy interchangeability of the sensor suite.

The power source may also be a battery. Other power sources for the devices 100, 200, 300 are possible, such as electrical outlets or solar. It is possible for a device to have the capability of being directly attached into an electrical power source. For example, as shown in FIG. 5C (showing the back panel and connection points) and FIG. 6, the AQOM device 300, has the capability of attaching directly to a junction box or power outlet.

In the embodiments that source power from an electrical, solar, or source other than a battery, the device can use the battery as a back-up or fail-safe source. In other words, should the main power source fail, the device could automatically source its power from the battery.

Interchangeable sensor suites 220 may be configured to target specific markets or consumers. For example, a sensor suite may focus on toxic gases that may then in turn inform other safety concerns. A sensor suite focused on toxic gases could include sensors that track and collect data on environmental variables like carbon monoxide, chloride, chlorine, hydrogen chloride, formaldehyde, nitrogen dioxide, ammonia, hydrogen sulfide, sulfur dioxide, hydrogen fluoride, phosphine, methane, etc.

In the embodiment shown in FIGS. 1A-1E and 2, the body 110 is comprised of a top cap 110a, a cylinder 110b, and a bottom cap 110c. The top cap 110a and bottom cap 110c may be configured with openings 112 to allow for air flow through the device 100 such that the sensors 122 are able to collect data from the air flow. The openings 112 may be mesh, slots, holes, or any other type of gap or opening.

In the embodiment shown in FIGS. 3A-3G and 4 the device 200 has openings 212 that allow for air flow through the device such that the sensors 222 in the sensor suite 220 are able to collect data from the air flow.

To be clear, an AQOM device 100, 200, 300 according to the present disclosure may have one sensor 122, 122. But, an AQOM device 100, 200, 300 according to the present disclosure may also have more than one sensor 122, 222. A device 100, 200, 300 may have 2, 3, 4, 5, or more sensors 122, 222.

In a preferred embodiment, the batteries 130, 230 are rechargeable. In some embodiments, recharging may occur via connecting the device 100, 200, 300 to an electrical outlet. In other embodiments, recharging may occur by removing or disconnecting the batteries 130, 230 from the device 100, 200, 300 and utilizing a separate battery charger. However, other powering and recharging methods are possible such as solar.

The sensors 122, 222 measure and collect various environmental information, such as temperature, humidity, CO2, volatile organic compounds, particulate matter, light, proximity, acoustic levels, or vibration. It is understood that other environmental information may be collected by the sensors. The rate at which information is measured and collected by the sensors 122, 222 can be adjusted (i.e., a variable data acquisition rate).

The sensors 122, 222 may have different ranges for each measurable variable. For example, the sensors may measure volatile organic compounds from 0 to 60,000 parts per billion (ppb) or more. For CO₂ measurements, the sensors may have the capability of measuring from 400 to 60,000 parts per million (ppm), or 300 to 60,000 ppm, 200 to 60,000 pm, etc. For temperature, the sensors may have the capability of measuring from −30 to 140° F. For relative humidity, the sensors may have the capability of measuring from 0.1% to 100%.

The AQOM device according to the present disclosure can take many different shapes, sizes, and configurations. Some embodiments may have a modular body (one example shown in FIGS. 3A-3G and 4) that allows for interchangeability of sensor suites communicatively connected to the printed circuit boards. The body of a modular AQOM device is configured to provide easy access to the printed circuit board and the sensors, such as through an access door, a clam shell type opening, and so on. Modular embodiments may house the printed circuit board and sensor suites in an interchangeable cartridge.

AQOM devices 100, 200, 300 according to the present disclosure are easily mountable to walls, desks, ceilings, or other furniture or surfaces. Some may be configured with a mounting component 150 as shown in FIGS. 1A-1E and 2. As discussed above, the devices 300 may be directly mounted to junction boxes or outlets.

They are also made from lightweight, low-cost material such as plastic or aluminum. Some embodiments may feature a clear body or an opaque body. AQOM devices according to the present disclosure may also feature indicators directly on or in the interior of the devices to provide information about the device such as, status (power status/level, on/off) and/or air quality. Other messaging via the indicators are possible. Indicators may be in the form of LEDs or a small screen on the device. If LEDs, the indicators can provide different indications or statuses using different colors or blinking.

The AQOM devices 100, 200, 300 may be used indoors or outdoors. An AQOM device according to the present disclosure for outdoor use would include a protective shield or mechanism attached to the device to create a barrier between the device and harmful elements, such as rain, moisture, and dust. The protective shield or mechanism could be as simple as adding additional mesh to the interior of the device or an umbrella-like covering on the outside and at the top of the device.

### The AQOM Portal Interface

AQOM devices 100, 200, 300 according to the present disclosure may connect and send information collected to a software application (i.e., a “portal”). The portal may be downloadable and usable on mobile electronics such as a tablet, laptop, or cell phone. The portal may also be accessible on through a web browser. The connection mechanism between AQOM devices 100, 200, 300 and the portal may be wired or wireless.

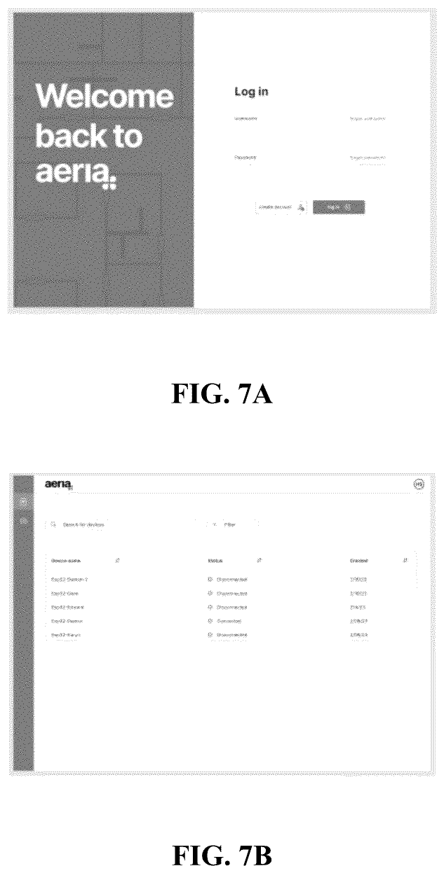

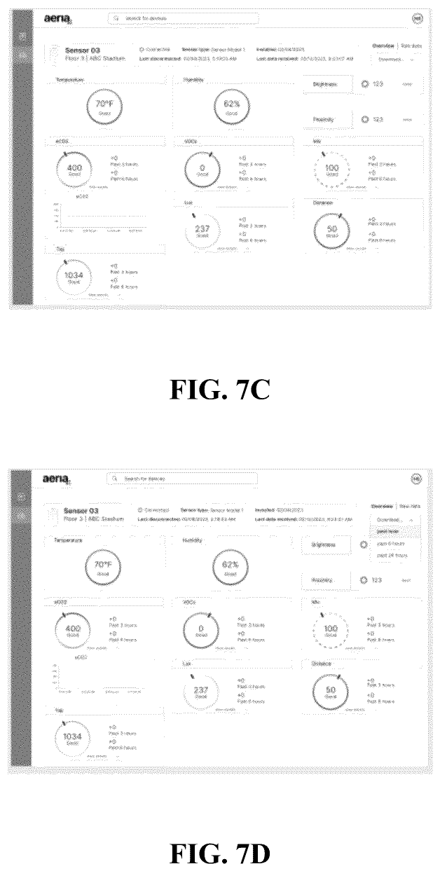

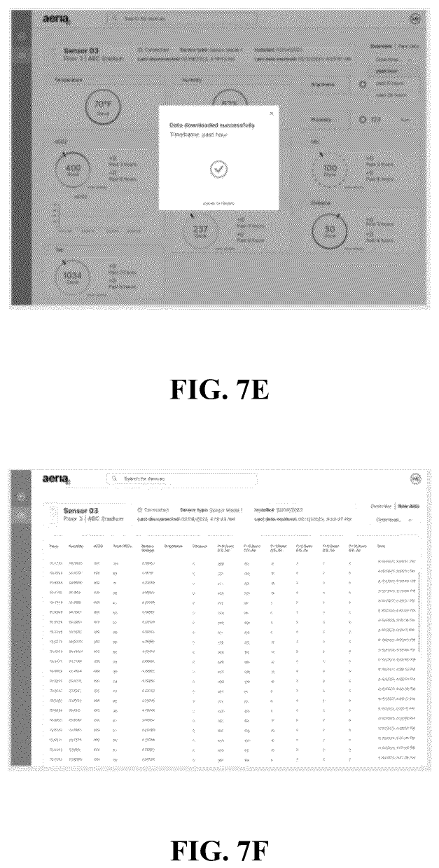

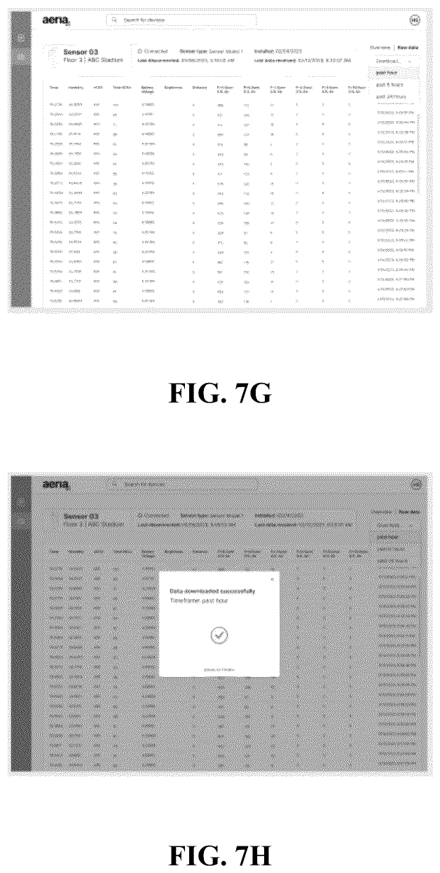

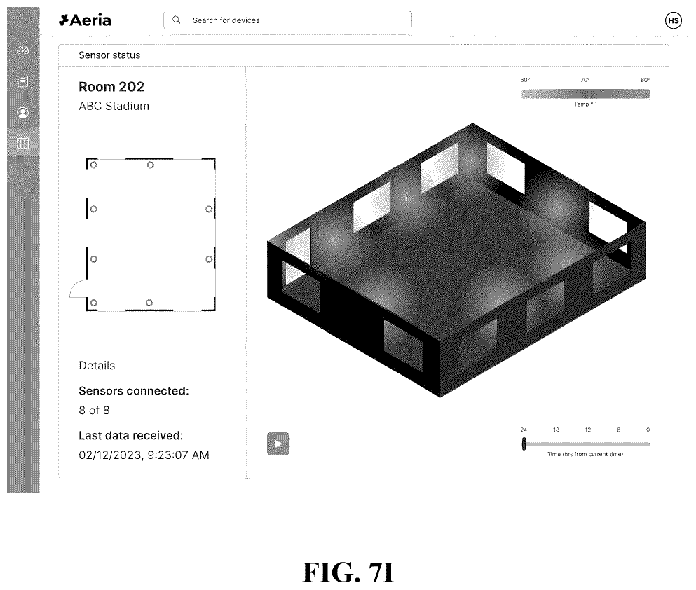

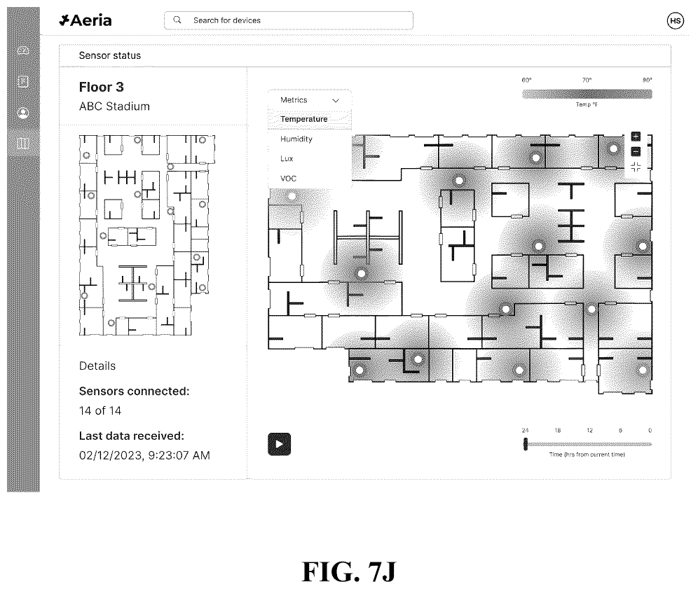

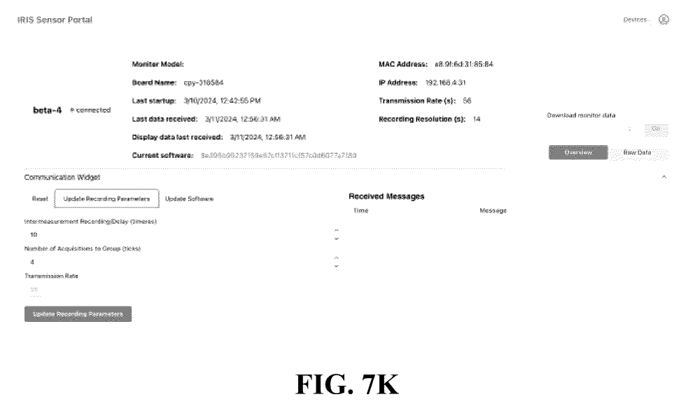

FIGS. 7A-7K shows one embodiment of a portal according to the present disclosure. FIGS. 7A-7K shows an example of one embodiment of a web-based portal that allows a user to:
Create an account and login (FIG. 7A);
View the live and historical status of any deployed AQOM devices (FIGS. 7B, 7C, and 7F);
View and download live or historical data from the AQOM devices (FIGS. 7D, 7E, 7G, and 7H);
View visualizations created by the portal (using inputted floor plan data) that allow a user to easily analyze temperature, humidity, air flow, occupancy patterns, and other data (FIGS. 71 and 7J); and
Send software updates to and/or communicate with the AQOM devices (FIG. 7K).

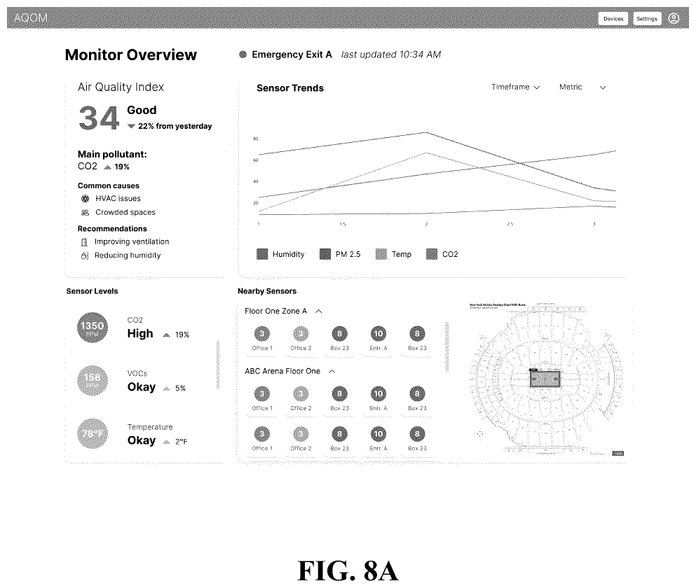

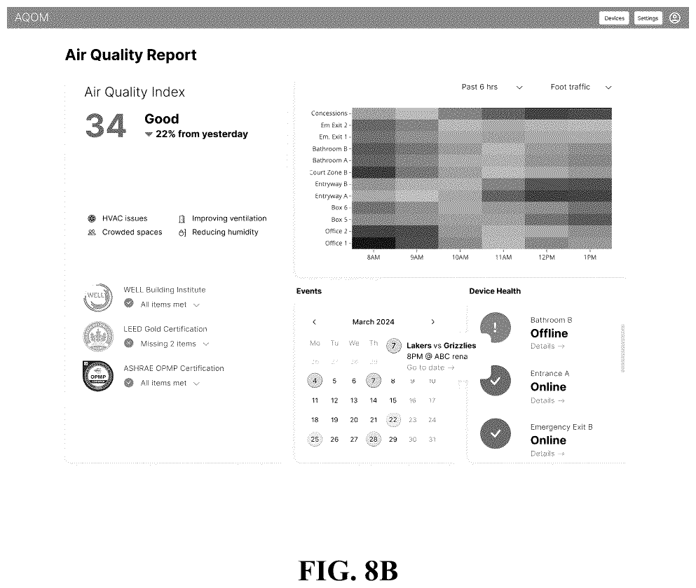

FIGS. 8A and 8B show another embodiment of possible dashboards or interfaces of the portal according to the present disclosure. It is understood that a portal according to the present disclosure will have different and/or other pages and capabilities than shown in FIGS. 7A-7K and specifically described above. FIGS. 7A-7K are examples of what one embodiment of a web portal may look like.

In some embodiments, the portal may further comprise a web-based or downloadable tool whereby a user may perform more complex data analysis, such as moving averages of frequency filters.

### The AQOM System

In one embodiment, an AQOM system comprises (1) at least one monitoring device 100, 200, 300 with at least one sensor 122, 222 capable of collecting environmental data and (2) a portal that communicates with the monitoring devices 100, 200, 300. The portal allows a user to access the environmental data collected by the monitoring devices. In a preferred embodiment, the portal and monitoring devices 100, 200, 300 wirelessly communicate. However, the portal and monitoring devices 100, 200, 300 may also communicate via a wired connection.

A system according to the present disclosure can have one AQOM device 100, 200, 300. A system may also have more than one AQOM device 100, 200, 300. If there is more than one AQOM device 100, 200, 300, they may communicate with one another via wireless or wired methods.

The number of devices in a system is generally dependent on the area that is to be monitored and the desired accuracy. A system according to the present disclosure has the ability to capture environmental data for single rooms, entire floors of buildings or structures, multiple floors, or entire buildings or structures. Similarly, if a plurality of devices 100, 200, 300 are deployed, different embodiments of the device 100, 200, 300 may be used in a system according the present disclosure, or the same embodiment may be utilized.

As noted above, the wireless communication between the AQOM devices 100, 200, 300 and the portal can be established over a wireless network. The network can be established via a modem and router(s), or it can be a mesh network (e.g., Zigbee, Thread, or Z-Wave) though it is understood that other modes of wireless communication may be used, such as radio frequency technology (e.g., Bluetooth or 900 Mhz Lora).

The AQOM devices disclosed herein may also report data to a server or gateway. The server or gateway can then report data via cellular modem where data will be stored—for example, to cloud storage. The data may also be stored locally.

FIGS. 5A-5K show one embodiment of a portal according to the present disclosure. Through the portal, a user will have access to view data and information collected by the sensors in the AQOM devices. In some embodiments of a portal according to the present disclosure, a user may view specific information about each AQOM device, such as location, status (e.g., connected, disconnected), MAC address, board name, IP address, firmware version, and historical and live data received from that device. In some embodiments of a portal according to the present disclosure, a user may download present or historical data from a particular AQOM device. A user may also be able to push software and other electrical updates or upgrades to one or all deployed AQOM device(s) through the portal.

In other embodiments of a portal according to the present disclosure, the portal may create various models and reports using the data and information collected by deployed AQOM devices. These models may show temperature variations, aerial dynamics, contaminant flow, and air stagnation risks, among other things. FIG. 7K shows an example temperature model that one embodiment of a portal according to the present disclosure may generate.

As shown in FIGS. 8A-8B, the reports may show air quality index, sensor trends, recommendations for adjustments like improving ventilation and reducing humidity, levels of various data points, certification information, and so on. Custom estimated occupancy indexes may also be included as a reported metric. An air quality index may be custom based on sensor function of two or more sensors or may be sourced from accredited third-party sources (e.g., RESET). A custom estimated occupancy index would be based on sensor fusion of two or more sensors.

The AQOM systems and devices disclosed herein improve energy efficiency, threat response capabilities, and occupant safety and comfort. With respect to energy efficiency, the data and information collected and analyzed by the AQOM systems disclosed herein can be used to control and optimize third-party systems such as HVAC systems, automatic doors, lighting, ventilation, and air filters. This data can be used to generally inform ventilation systems or habits for maintaining optimum levels for occupants, staff, and/or personnel.

Active monitoring and optimization of this equipment will not only provide a financial return on investment for users, but can be considered positive climate preservation efforts. With respect to threat response capabilities, the AQOM systems disclosed herein provide a full layout of an area or venue to a user with information regarding associated hazardous risks. The systems can provide visual feedback during an emergency situation, which can in turn expedite and improve responder performance, as well as potentially provide predictive factors for emergency events. With respect to occupant safety and comfort, the AQOM systems disclosed herein collect air quality information such as carbon dioxide, particular matter, and VOCs levels. Having adequate carbon dioxide and particular matter monitoring is also important for respiratory health and cognitive function.

Another embodiment of an AQOM system according to the present disclosure further comprises at least one display for safety indicators or exit strategies. In some embodiments, the AQOM devices are connected to at least one display that may display different words or characters. For example, should the sensors in the AQOM devices within the system detect toxic air quality or an unhealthy environment, the display can show characters or words directing occupants towards the nearest exits in the occupant's vicinity. These words and characters could be: “<-EXIT<-,” “\hat{ }EXIT\hat{ },!” “NO EXIT,” or “->EXIT->.” The display could also show warning words or instructions such as “TOXIN DETECTED” or “ALL CLEAR” or “ALL OK!” It is known that many combinations of words, characters, symbols, and/or language may be shown on the display. The AQOM device, portal, and displays are all communicatively connected to one another through wireless communication networks. These embodiments may also include the ability of the system to develop routines to identify exit routes for a designated area or space.

An AQOM system according to the present disclosure may also comprise a software application for occupants other than managers to provide occupants access to limited amounts of information gathered by the AQOM devices. This software application may be configured for phones or computers. One of the purposes of this application would be to give the system and the managers of the system the ability to send instant alerts to occupants. The application may provide push notifications to occupants' mobile phones and instructions on the nearest exits.

The AQOM systems and devices disclosed herein can be used in small or large environments, for example, hospitals, large event venues, military outfits and bases, certification organizations, science and engineering labs, manufacturing plants, power plants, and business offices. However, the systems and devices can also be used in smaller areas like a residential home or apartment.

We claim:

1. An air quality and occupancy monitoring (AQOM) system comprising:
at least one monitoring device comprised of at least one sensor configured to collect environmental data; and
a portal configured to communicate with said at least one monitoring device and allow a user to access said environmental data collected from said at least one monitoring device.

2. The AQOM system of claim 1, wherein said at least one monitoring device is powered by a removable battery.

3. The AQOM system of claim 2, wherein said removable battery is also rechargeable.

4. The AQOM system of claim 1, wherein said at least one monitoring device further comprises a battery as an automatic alternative power source should the electrical source fail.

5. The AQOM system of claim 3, further comprising a battery charging device configured to recharge said rechargeable batteries for said at least one monitoring device.

6. The AQOM system of claim 1, wherein said device further comprises an interchangeable component configured with said at least one sensor.

7. The AQOM system of claim 1, wherein live environmental data from said at least one monitoring device may be accessed using said portal.

8. The AQOM system of claim 1, wherein historical environmental data from said at least one monitoring device may be accessed from said portal.

9. The AQOM system of claim 1, wherein said portal is web-based.

10. The AQOM system of claim 1, wherein said portal is a downloadable software application.

11. The AQOM system of claim 1, further comprising at least one display configured to receive and show information from said at least one monitoring device.

12. The AQOM system of claim 1, further comprising at least one display configured to receive and show information from said portal.

13. An air quality and occupancy monitoring (“AQOM”) device comprising:
a body;
a printed circuit board;
at least one sensor configured to measure and collect environmental information; and
a power source configured to connect to said body, said printed circuit board, and said at least one sensor.

14. The AQOM device of claim 13, wherein said power source is a removable battery.

15. The AQOM device of claim 13, wherein said battery is rechargeable.

16. The AQOM device of claim 13, further comprising an interchangeable component configured with said at least one sensor.

17. The AQOM device of claim 13, wherein said device is configured to wirelessly send said environmental information to a portal.

18. The AQOM device of claim 17, wherein said device is further configured to wirelessly send information concerning said device to said portal.

19. The AQOM device of claim 17, wherein said device is further configured to wirelessly receive software updates from said portal.

20. The AQOM device of claim 13, further comprising at least one indicator.

21. The AQOM device of claim 20, wherein said at least one indicator is a light emitting diode.

22. The AQOM device of claim 20, wherein said at least one indicator is a display.
